(19) United States
(12) Patent Application Publication
(10) Pub. No.: US 2016/0051285 A1
(43) Pub. Date: Feb. 25, 2016

(54) CONNECTING ROD FOR SPINAL SURGERY

(71) Applicant: Spineology Inc., St. Paul, MN (US)

(72) Inventor: Joe Gleason, Eagan, MN (US)

(21) Appl. No.: 14/832,998

(22) Filed: Aug. 21, 2015

Related U.S. Application Data
(60) Provisional application No. 62/040,409, filed on Aug. 21, 2014.

(51) Int. Cl. A61B 17/70

(52) U.S. Cl. CPC A61B 17/7004; A61B 17/7032; A61B 2017/564

(57) ABSTRACT

An interconnecting spinal rod with a flanged end permits the surgeon to specifically locate the superior end, screw/rod position thereby minimizing the connecting rod overhang. This minimal overhang reduces interference with the adjacent facet of a vertebra in a spinal interbody fusion surgical procedure.

### PRIORITY

This application claims the benefit of U.S. Provisional Application Ser. No. 62/040,409, filed on Aug. 21, 2014, which is hereby incorporated herein by reference in its entirety.

### FIELD

The present invention generally relates to connecting screw rods. More particularly, the present invention relates to a pedicle screw interconnecting rod for use in spine surgery.

### BACKGROUND

In spinal surgical procedures, pedicle screw constructs use multiple screws nested along a connecting rod to anchor spinal vertebral bodies posteriorly as adjunct fixation to interbody fusion surgical procedures. The rod lengths are chosen through measuring devices which usually add additional overhang length on each end of the construct to ensure full engagement within the pedicle screw saddle on each end.

When the pedicle screws are implanted in a sagittal plane, aligned with the facet joint of the spine, the superior/caudal (head) screw is implanted immediately below the intact, mobile facet joint. To prevent collision of the connecting rod with this mobile facet joint, the surgeon's goal is to minimize the connecting rod overhang at that pedicle screw.

### SUMMARY

The disclosure includes an interconnecting spinal rod with a flanged end to specifically locate the superior end, screw/rod position minimizing the connecting rod overhang. This minimal overhang reduces interference with the adjacent facet in spinal interbody fusion surgical procedures.

The disclosure includes a bony fixation system. The system includes a first tulip head screw and a second tulip head screw. A connecting rod including a flanged end is disposed in a portion of each of the first and second tulip head screws such that the flanged end abuts the first tulip head screw. A first screw is disposed in the first tulip head screw. A second screw is disposed in the second tulip head screw.

The disclosure further includes a method of bony fixation. The method includes placing a cephalad tulip head screw into a bone and placing a caudad tulip head screw into the bone. Then a connecting rod having a flanged end is advanced into the first and second tulip heads. The rod is moved caudally until the flanged end abuts the cephalad tulip head. Set screws are secured into each of the tulip head screws.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a perspective assembly view of a screw, cap, tulip and connecting rod according to an embodiment of the present invention.

FIG. 2 is another perspective view of a screw, cap, tulip and connecting rod assembly according to an embodiment of the present invention.

FIG. 3 is another perspective view of a screw, cap, tulip and connecting rod assembly according to an embodiment of the present invention.

FIG. 4 is a perspective view of a pedicle screw and connecting rod construct in a spinal interbody fusion surgical procedure according to an embodiment of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

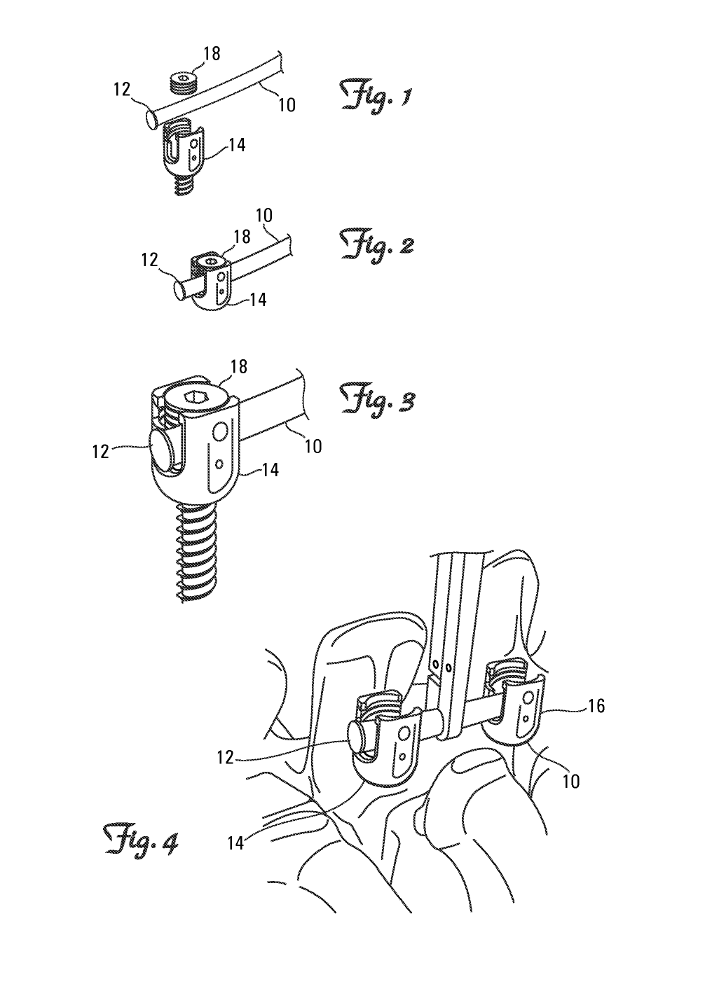

As is depicted in FIGS. 1-3, connecting rod 10 may include a flanged or “T-shaped” end 12. The flange diameter defines a larger diameter than the rod diameter. The flange abuts a tulip head 14 of a screw as shown in FIGS. 3-4.

In use, the connecting rod 10 may be longitudinally advanced until it is seated in the two or more tulip heads. Connecting rod 10 may then be pulled caudally until flange 12 abuts a first cephalad tulip head 14, as shown in FIGS. 3 and 4, ensuring correct rod placement. A set screw 18 may then be secured into place in each of the tulip heads.

A recess or groove can be defined in the circumferential outside surface of the tulip to receive the flanged end 12. A flat section of the outer tulip circumferential surface can also be defined to enhance stability of the flanged end engaging the tulip.

Abutting flange 12 against tulip head 14 reduces the potential for adjacent facet encroachment. Flange 12 directs rod placement such that the rod is automatically positioned in the correct location between the at least two tulip heads, for example as shown in FIG. 4, tulip head 14 and tulip head 16.

The disclosures of U.S. Pub. Pat. App. Nos. 2013/0110124 and 2015/0105832 are both hereby incorporated herein by reference in their entirety.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A bony fixation system comprising:
a first tulip head screw;
a second tulip head screw;
a connecting rod including a flanged end disposed in a portion of each of the first and second tulip head screws, wherein the flanged end abuts the first tulip head screw;
a first screw disposed in the first tulip head screw; and
a second screw disposed in the second tulip head screw.

2. A method of bony fixation comprising:
placing a cephalad tulip head screw into a bone;
placing a caudad tulip head screw into the bone;
advancing a connecting rod having a flanged end into the first and second tulip heads;
moving the rod caudally until the flanged end abuts the cephalad tulip head; and
securing a set screw into each of the tulip head screws.
